(19) United States
(12) Patent Application Publication
(10) Pub. No.: US 2007/0154454 A1
(43) Pub. Date: Jul. 5, 2007

(54) Compositions and methods for treating poxvirus infection

(76) Inventors: Brian Murphy, Newport Coast, CA (US); Lawrence M. Blatt, San Francisco, CA (US)

Correspondence Address: THELEN REID BROWN RAYSMAN & STEINER LLP, 900 THIRD AVENUE, NEW YORK, NY, 10022, US

(21) Appl. No.: 10/551,368

(22) PCT Filed: Mar. 11, 2004
(86) PCT No.: PCT/US04/07509

§ 371 Date: Oct. 11, 2006

Related U.S. Application Data
(60) Provisional application No. 60/458,916, filed on Mar. 28, 2003.

(51) Int. Cl. A61K 38/21; A61K 39/275

(52) U.S. Cl. 424/85.7; 424/232.1

(57) ABSTRACT

The present invention provides methods of treating a poxvirus infection, e.g., a vaccinia virus or a variola virus infection; and methods of reducing viral load, or reducing the time to viral clearance, or reducing morbidity or mortality in the clinical outcomes, in patients suffering from a poxviral infection. The present invention further provides methods of reducing the risk that an individual will develop a pathological poxvirus infection, such as a vaccinia virus infection or a variola virus infection, that has clinical sequelae. The methods generally involve administering a therapeutically effective amount of a Type I or Type III interferon receptor agonist and/or a Type II interferon receptor agonist for the treatment of a pox viral infection.

### FIELD OF THE INVENTION

The present invention is in the field of poxvirus infections, especially the treatment of variola virus and vaccinia virus infection.

### BACKGROUND OF THE INVENTION

The smallpox (variola) virus is a poxvirus, a member of the Poxyiridae family of viruses which includes Orthopoxvirus such as variola (smallpox) and vaccinia; Avipoxvirus such as fowlpox; Capripoxvirus; Leoporipoxvirus; Parapoxvirus; Molluscipoxvirus; and Yatapoxvirus. Poxviruses are characterized by having a large, complex virion containing enzymes that synthesize mRNA; a genome composed of a single linear double-stranded DNA molecule of 130-300 kilobase pairs with a hairpin loop at each end; and a cytoplasmic site of replication.

Smallpox is a serious, contagious, and sometimes fatal infectious disease. There is currently no specific treatment for smallpox disease, and the only prevention is vaccination, or isolation of at-risk populations. Because virulent smallpox strains appear to have no animal reservoir, and because vaccination using vaccinia virus is very effective in inducing protective immunity to variola virus, this virus has been virtually eliminated from the world. Despite eradication of the wild-type virus, there exists the possibility of populations being exposed to stored wild-type or genetically altered variola virus. In view of the potential risk, certain segments of the population may be vaccinated with vaccinia virus.

Generally, the smallpox vaccine is safe and effective. However, in the past, approximately 14 to 52 people in every 1 million vaccinated for the first time developed clinical infection with the virus, e.g., viral replication was not limited by a protective immune response in these individuals. Indeed, based on past experience, it is estimated that between 1 and 2 people out of every 1 million vaccinated may die as a result of vaccinia virus infection.

Furthermore, among individuals who do not have protective immunity to smallpox virus, the overall fatality rate from smallpox infection is estimated to be approximately 30%.

In view of the potential for serious to life-threatening pathological infection with vaccinia virus following vaccine, and in view of the high mortality rate associated with smallpox virus in individuals lacking protective immunity, there is a need in the art for methods for reducing the risk of pathological infection, and for methods of treating an active pathological infection in individuals infected with poxvirus such as vaccinia virus or smallpox virus. The present invention addresses these needs.

### SUMMARY OF THE INVENTION

The present invention provides methods of treating a poxvirus infection, e.g., a vaccinia virus or a variola virus infection; and methods of reducing viral load, or reducing the time to viral clearance, or reducing morbidity or mortality in the clinical outcomes, in patients suffering from a poxviral infection. The present invention further provides methods of reducing the risk that an individual will develop a pathological poxvirus infection, such as a vaccinia virus infection or a variola virus infection, that has clinical sequelae. The methods generally involve administering a therapeutically effective amount of a Type I or Type III interferon receptor agonist and/or a Type II interferon receptor agonist for the treatment of a pox viral infection.

### FEATURES OF THE INVENTION

The invention features a method of treating a pox viral infection, generally involving administering to an individual a) a Type II interferon receptor agonist; b) a Type I or Type III interferon receptor agonist; or c) a Type II interferon receptor agonist and a Type I or Type III interferon receptor agonist concurrently, in an amount effective to ameliorate the clinical course of the disease. The invention also features a method of treating a pox viral infection by administering to an individual a Type II interferon receptor agonist and a Type I or Type III interferon receptor agonist in a synergistically effective amount to ameliorate the clinical course of the disease.

The invention features a method of treating a vaccinia viral infection, generally involving administering to an individual a Type I or Type III interferon receptor agonist, a Type II interferon receptor agonist, or a Type II interferon receptor agonist and a Type I or Type III interferon receptor agonist concurrently, in an amount effective to reduce the time to viral clearance or to reduce morbidity or mortality in clinical outcomes. The invention also features a method of treating a vaccinia viral infection by administering to an individual a Type II interferon receptor agonist and a Type I or Type III interferon receptor agonist in a synergistically effective amount to reduce the time to viral clearance or to reduce morbidity or mortality in clinical outcomes.

The invention features a method of treating a smallpox viral infection, generally involving administering to an individual a Type I or Type III interferon receptor agonist, a Type II interferon receptor agonist, or a Type II interferon receptor agonist and a Type I or Type III interferon receptor agonist concurrently, in an amount effective to reduce the time to viral clearance or to reduce morbidity or mortality in clinical outcomes. The invention also features a method of treating a smallpox viral infection by administering to an individual a Type II interferon receptor agonist and a Type I or Type III interferon receptor agonist in a synergistically effective amount to reduce the time to viral clearance or to reduce morbidity or mortality in clinical outcomes.

In carrying out the methods of combination therapy for a poxviral infection, vaccinia viral infection, or smallpox viral infection in an individual as described above, a Type I or Type III interferon receptor agonist and a Type II interferon receptor agonist are administered to the individual. In some embodiments, the Type I or Type III interferon receptor agonist and the Type II interferon receptor agonist are administered in the same formulation. In other embodiments, the Type I or Type III interferon receptor agonist and the Type II interferon receptor agonist are administered in separate formulations. When administered in separate formulations, a Type I or Type III interferon receptor agonist and a Type II interferon receptor agonist can be administered substantially simultaneously, or can be administered within about 24 hours of one another. In many embodiments, a Type I or Type III interferon receptor agonist and a Type II interferon receptor agonist are administered subcutaneously in multiple doses. Optionally, the Type I or Type III interferon receptor agonist and/or the Type II interferon receptor agonist is administered to the individual by a controlled drug delivery device. Optionally, the Type I or Type III interferon receptor agonist and/or the Type II interferon receptor agonist is administered to the individual substantially continuously or continuously by a controlled drug delivery device. Optionally, the controlled drug delivery device is an implantable infusion pump and the infusion pump delivers the Type I or Type III interferon receptor agonist and/or the Type II interferon receptor agonist to the individual by subcutaneous infusion.

In some embodiments utilizing combination therapy, the Type II interferon receptor agonist is administered during the entire course of Type I or Type III interferon receptor agonist treatment. In other embodiments, the Type II interferon receptor agonist is administered for a period of time that is overlapping with that of the Type I or Type III interferon receptor agonist treatment, e.g., the Type II interferon receptor agonist treatment can begin before the Type I or Type III interferon receptor agonist treatment begins and end before the Type I or Type III interferon receptor agonist treatment ends; the Type II interferon receptor agonist treatment can begin after the Type I or Type III interferon receptor agonist treatment begins and end after the Type II interferon receptor agonist treatment ends; the Type II interferon receptor agonist treatment can begin after the Type I or Type III interferon receptor agonist treatment begins and end before the Type I or Type III interferon receptor agonist treatment ends; or the Type II interferon receptor agonist treatment can begin before the Type I or Type III treatment begins and end after the Type I or Type III interferon receptor agonist treatment ends.

In some embodiments, Type I or Type III interferon receptor agonist treatment, Type II interferon receptor agonist treatment, or combination therapy with a Type I or Type III interferon receptor agonist and a Type II interferon receptor agonist is carried out with co-administration of an additional specific antiviral agent that is effective against poxvirus infection. In these embodiments, the duration of therapy with the additional antiviral agent can be coincident with the duration of therapy with a Type I or Type III interferon receptor agonist and/or a Type II interferon receptor agonist. In other embodiments, the course of therapy with the additional antiviral agent can overlap with the course of therapy with a Type I or Type III interferon receptor agonist and/or a Type II interferon receptor agonist, e.g., the additional antiviral agent treatment can begin before the treatment with Type I or Type III interferon receptor agonist and/or Type II interferon receptor agonist begins and end before treatment with Type I or Type III interferon receptor agonist and/or Type II interferon receptor agonist ends; the additional antiviral agent treatment can being after the treatment with Type I or Type III interferon receptor agonist and/or Type II interferon receptor agonist begins and end before the treatment with Type I or Type III interferon receptor agonist and/or Type II interferon receptor agonist ends; or the additional antiviral agent treatment can begin before the treatment with Type I or Type III interferon receptor agonist and/or Type II interferon receptor agonist begins and end after treatment with Type I or Type III interferon receptor agonist and/or Type II interferon receptor agonist ends.

In other embodiments, the Type I or Type III interferon receptor agonist treatment, Type II interferon receptor agonist treatment, or combination therapy with a Type I or Type III interferon receptor agonist and a Type II interferon receptor agonist, is carried out in conjunction with administration of a vaccinia virus vaccine. In some of these embodiments, the interferon therapy is administered within 7 to 10 days before the vaccinia virus vaccine is administered. In other embodiments, the interferon therapy is administered within 7 to 10 days after the vaccinia virus vaccine is administered. In other embodiments, the interferon therapy is co-administered (e.g., administered concurrently with) with the vaccinia virus vaccine.

In many embodiments, any of the above-described methods involve administering IFN-γ. In some of these embodiments, the methods involve co-administering vaccinia virus vaccine and IFN-γ in the prophylactic or therapeutic treatment of a smallpox viral infection in a patient In other embodiments, the IFN-γ therapy or the vaccinia virus vaccine and IFN-γ combination therapy is co-administered with one or more additional antiviral agent(s) that is effective in treating a poxvirus infection.

In many embodiments, any of the above-described methods involve administering IFN-α. In some of these embodiments, the methods involve co-administering vaccinia virus vaccine and IFN-α in the prophylactic or therapeutic treatment of a smallpox viral infection in a patient. In other embodiments, the IFN-α therapy or the vaccinia virus vaccine and IFN-α combination therapy is co-administered with one or more additional antiviral agent(s) that is effective in treating a poxvirus infection.

In many embodiments, any of the above-described methods involve administering consensus interferon (CIFN). In some of these embodiments, the methods involve co-administering vaccinia virus vaccine and CIFN in the prophylactic or therapeutic treatment of a smallpox viral infection in a patient. In other embodiments, the CIFN therapy or the vaccinia virus vaccine and CIFN combination therapy is co-administered with one or more additional antiviral agent(s) that is effective in treating a poxvirus infection.

In many embodiments, any of the above-described methods involve administering IFN-α and IFN-γ. In some of these embodiments, the methods involve co-administering vaccinia virus vaccine, IFN-α, and IFN-γ. In other embodiments, the methods involve co-administering IFN-α, IFN-γ, and an additional antiviral agent that is effective in treating a poxvirus infection.

In many embodiments, any of the above-described methods involve administering CIFN and IFN-γ. In some of these embodiments, the methods involve co-administering vaccinia virus vaccine, CIFN, and IFN-γ. In other embodiments, the methods involve co-administering CIFN, IFN-γ, and an additional antiviral agent that is effective in treating a poxvirus infection.

In many embodiments, any of the above-described methods involve administering a PEGylated IFN-α conjugate. In some embodiments, the PEGylated IFN-α conjugate is a monoPEGylated IFN-α. In other embodiments, the monoPEGylated IFN-α conjugate is an IFN-α polypeptide covalently linked to a single PEG moiety via a lysine residue or the N-terminal amino acid residue of the IFN-α polypeptide. In other embodiments, the monoPEGylated IFN-α conjugate is an IFN-α polypeptide covalently linked to a single PEG moiety via an amide bond between either the epsilon-amino group of a lysine residue or the alpha-amino group of the IFN-α polypeptide and an activated carboxyl group of the PEG moiety. In other embodiments, the monoPEGylated IFN-α conjugate is an IFN-α polypeptide covalently linked to a single, linear PEG moiety. In other embodiments, the monoPEGylated IFN-α conjugate is an IFN-α polypeptide covalently linked to a single, linear 30 kD PEG moiety. In other embodiments, the monoPEGylated IFN-α conjugate is an IFN-α polypeptide covalently linked to a single, linear 30 kD PEG moiety via an amide bond between the epsilon-amino group of a lysine residue or the alpha-amino group of the IFN-α polypeptide and an activated carboxyl group of the PEG moiety. In other embodiments, the monoPEGylated IFN-α conjugate is an IFN-α polypeptide covalently linked to a single, linear 30 kD PEG via an amide bond between the epsilon-amino group of a lysine residue or the alpha-amino group of the IFN-α polypeptide and an activated propionyl group of the PEG moiety. In other embodiments, the monoPEGylated IFN-α conjugate is an IFN-α polypeptide covalently linked to a single, linear monomethoxy-PEG (mPEG). In other embodiments, the monoPEGylated IFN-α conjugate is the product of a condensation reaction between an IFN-α polypeptide and a linear, succinimidyl propionate ester-activated 30 kD mPEG. In any of the foregoing methods using a PEGylated IFN-α conjugate, the IFN-α polypeptide can be a consensus interferon (CIFN) polypeptide. In any of the foregoing methods using a PEGylated IFN-α conjugate, the IFN-α polypeptide can be a CIFN polypeptide that is interferon alfacon-1.

### Definitions

The terms “individual,” “host,” “subject,” and “patient” are used interchangeably herein, and refer to a mammal, including, but not limited to, primates, including simians and humans.

As used herein, the term “poxvirus” includes any member of the family Poxyiridae, including, but not limited to, any member of the Orthopoxvirus genus, including variola (smallpox) virus, vaccinia virus, camelpox, cowpox, ectromelia, monkeypox, racoonpox, skunkpox, taterapox, Uasin Gishu, and volepox; any member of the Parapoxvirus genus; any member of the Avipoxvirus genus; any member of the Capripoxyinus genus; any member of the Leporipoxvirus genus; any member of the Suipoxvirus genus; any member of the Molluscipoxvirus genus; any member of the Yatapoxvirus genus; and any member of the Entomopoxvirus A, B, or C genus.

The term “poxvirus” further includes naturally-occurring (e.g., wild-type) poxvirus; naturally-occurring poxvirus variants; and poxvirus variants generated in the laboratory, including variants generated by selection, variants generated by chemical modification, and genetically modified variants (e.g., poxvirus modified in a laboratory by recombinant DNA methods).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “a Type III interferon receptor agonist” refers to any naturally occurring or non-naturally occurring ligand of humanIL-28 receptor α (“IL-28R”), the amino acid sequence of which is described by Sheppard, et al., infra., that binds to and causes signal transduction via the receptor.

As used herein, the term “a Type II interferon receptor agonist” refers to any naturally-occurring or non-naturally-occurring ligand of a human Type II interferon receptor which binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

The term “dosing event” as used herein refers to administration of an antiviral agent to a patient in need thereof, which event may encompass one or more releases of an antiviral agent from a drug dispensing device.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance (e.g., a Type I or Type III interferon receptor agonist, e.g., IFN-α; e.g., a Type II interferon receptor agonist, e.g., IFN-γ) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a dose” includes a plurality of such doses and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating a poxvirus infection, e.g., a vaccinia virus or a variola virus infection; and methods of reducing viral load, or reducing the time to viral clearance, or reducing morbidity or mortality in the clinical outcomes, in patients suffering from a pox viral infection. The present invention further provides methods of reducing the risk that an individual will develop a pathological poxvirus infection, such as a vaccinia virus infection or a variola virus infection, that has clinical sequelae. The methods generally involve administering a therapeutically effective amount of a Type I or Type III interferon receptor agonist and/or a Type II interferon receptor agonist for the treatment of a pox viral infection.

The methods of the present invention generally involve administering: a) a Type II interferon receptor agonist; b) a Type I or Type III interferon receptor agonist; of c) a Type II interferon receptor agonist and a Type I or Type III interferon receptor agonist concurrently. For convenience, treatments a), b), and c) are collectively referred to herein as “interferon therapy” or “interferon treatment.”

In the description provided herein, vaccinia virus and smallpox virus are exemplified. However, the subject methods are useful for the treatment, including prophylaxis, of any poxvirus infection.

When vaccinia virus vaccine is administered to individuals with a normal, intact immune system (e.g. individuals who are not immunocompromised), an infection ensues. Such an infection is generally beneficial, as it provides protective immunity to smallpox virus. Protective immunity typically is produced within 7 to 10 days following vaccination. However, in some of these individuals, the infection is not limited by an immune response induced in the individual, and a pathological infection with clinical sequelae, i.e., a disease state, ensues. The present invention provides methods for treating a pathological vaccinia virus infection, e.g., a vaccinia virus infection with clinical sequelae, e.g., vaccinia virus disease. A pathological vaccinia virus infection is indicated by one or more of the following: a temperature often exceeding 40° C.; a temperature often exceeding 40° C. accompanied by malaise, headache, and body aches; vaccinia rash; and the like.

In some embodiments, the interferon therapy is administered to an individual prophylactically, e.g., interferon therapy is initiated before the administration of a vaccinia virus vaccine, concurrently with administration of a vaccinia virus vaccine, or within 7-14 days after administration of a vaccinia virus vaccine. An advantage of the subject methods is that the risk that an individual will develop a pathological vaccinia virus infection following vaccination with vaccinia virus is reduced.

In other embodiments, the interferon therapy is initiated after the appearance of clinical signs of a pathological vaccinia virus infection, e.g., the appearance of a fever often exceeding 40° C. within about 7 to 14 days following vaccinia virus vaccination. An advantage of the subject methods is that the severity of the pathological vaccinia virus infection is reduced, e.g., the viral load is reduced, and/or the time to viral clearance is reduced, and/or the morbidity or mortality is reduced.

The present invention further provides methods of treating a pathological vaccinia virus infection in individuals for whom the vaccinia virus vaccine is currently contraindicated. An advantage of the methods of the present invention is that individuals for whom vaccinia virus vaccination is currently not recommended may receive the vaccinia virus vaccine, and thus may develop protective immunity to smallpox virus. Treatment with a subject methods reduces the risk that such an individual will develop a pathological vaccinia virus infection. Individuals for whom vaccinia virus vaccination is currently contraindicated include individuals with eczema or atopic dermatitis; individuals with skin conditions such as burns, chickenpox, shingles, impetigo, herpes, severe acne, or psoriasis; individuals who are immunocompromised due to cancer chemotherapy, drugs to reduce organ rejection following organ transplant, human immunodeficiency virus infection, medications to treat autoimmune disorders, and the like; individuals with certain heart conditions (e.g., coronary artery disease); and women who are pregnant or who are planning to become pregnant within one month of vaccination.

The present invention further provides methods of prophylactically treating smallpox virus infection. In view of the high mortality rate associated with smallpox virus infection, any expected exposure, suspected exposure, or known exposure to smallpox virus is considered cause for initiating treatment with the methods of the present invention. An advantage of the subject methods is that prophylactic interferon treatment reduces the risk that an individual who has been exposed to smallpox virus will develop an infection with the virus and/or will exhibit clinical symptoms of smallpox virus infection. A further advantage of the subject methods is that prophylactic interferon treatment reduces the clinical symptoms of smallpox virus infection should such an infection occur.

The present invention further provides methods of therapeutically treating smallpox or vaccinia virus infection in individual who present with clinical signs of smallpox or vaccinia virus infection following known or suspected exposure to smallpox or vaccinia virus or following vaccination with vaccinia virus vaccine. Individuals (i) who have received vaccination with vaccinia virus vaccine or who have known or suspected exposure to smallpox or vaccinia virus and (ii) who present with a fever often exceeding 40° C. are considered eligible for treatment with the methods of the present invention. An advantage of the subject methods is that the severity of the smallpox or vaccinia virus infection is reduced, e.g., the viral load is reduced, and/or the time to viral clearance is reduced, and/or the morbidity or mortality is reduced.

Where a subject treatment method is prophylactic, the methods reduce the risk that an individual will develop pathological infection with a poxvirus, e.g., a vaccinia virus or a smallpox virus. Effective amounts of a Type I or Type III interferon receptor agonist or a Type II interferon receptor agonist are amounts that, alone or in combination therapy, reduce the risk or reducing the probability that an individual will develop a pathological infection with a poxvirus. For example, an effective amount reduces the risk that an individual will develop a pathological infection by at least about 10%, at least about 20%, at least about 25%, at least about 30%, at least about 35%, at least about 40%, at least about 50%, at least about 60%, at least about 70%, at least about 80%, at least about 90%, or more, compared to the risk of developing a pathological infection with the virus in the absence of interferon treatment.

In some embodiments, effective amounts of a Type I or Type III interferon receptor agonist or a Type II interferon receptor agonist are amounts that, alone or in combination therapy, reduce poxvirus viral load by at least about 10%, at least about 20%, at least about 25%, at least about 30%, at least about 35%, at least about 40%, at least about 50%, at least about 60%, at least about 70%, at least about 80%, at least about 90%, or more, compared to the viral load in the absence of treatment.

In some embodiments, effective amounts of a Type I or Type III interferon receptor agonist or a Type II interferon receptor agonist are amounts that, alone or in combination therapy, reduce the time to viral clearance, by at least about 10%, at least about 20%, at least about 25%, at least about 30%, at least about 35%, at least about 40%, at least about 50%, at least about 60%, at least about 70%, at least about 80%, at least about 90%, or more, compared to the time to viral clearance in the absence of treatment.

In some embodiments, effective amounts of a Type I or Type III interferon receptor agonist or a Type II interferon receptor agonist are amounts that, alone or in combination therapy, reduce morbidity or mortality due to a poxvirus infection by at least about 10%, at least about 20%, at least about 25%, at least about 30%, at least about 35%, at least about 40%, at least about 50%, at least about 60%, at least about 70%, at least about 80%, at least about 90%, or more, compared to the morbidity or mortality in the absence of treatment.

Whether a subject treatment method is effective in reducing the risk of a pathological poxvirus infection, reducing viral load, reducing time to viral clearance, or reducing morbidity or mortality due to a poxvirus infection is readily determined by those skilled in the art. Viral load is readily measured by measuring the titer or level of virus in serum. The number of virus in the serum can be determined using any known assay, including, e.g., a quantitative polymerase chain reaction assay using oligonucleotide primers specific for the poxvirus being assayed. Whether morbidity is reduced can be determined by measuring any symptom associated with a poxvirus infection, including, e.g., fever, the extent of rash formation, the number of pustules, and the like.

Generally, unit dosage forms of a Type I or Type III interferon receptor agonist range from about 1 μg to about 300 μg. Generally, unit dosage forms of a Type II interferon receptor agonist can range from about 25 μg to about 500 μg. In many embodiments, multiple doses of a Type I or Type III interferon receptor agonist and/or a Type II interferon receptor agonist will be administered. For example, a Type I or Type III interferon receptor agonist and/or a Type II interferon receptor agonist can be administered twice daily, daily, three times a week, twice a week, weekly, or twice a month for a period of one week to about 16 weeks.

In some embodiments, the present invention provides methods for reducing the risk that an individual will develop a pathological vaccinia virus infection in an individual who has been vaccinated with vaccinia virus vaccine. In some of these embodiments, the methods involve administering an effective amount of a Type I or a Type III interferon receptor agonist. In other embodiments, the methods involve administering an effective amount of a Type II interferon receptor agonist. In other embodiments, the methods involve administering effective amounts of a Type I or a Type III interferon receptor agonist in combination therapy with an effective amount of a Type II interferon receptor agonist. In some embodiments, interferon therapy is begun from one day to about 14 days, or from about one day to about 7 days before vaccination with vaccinia virus. In other embodiments, interferon treatment occurs on the same day as vaccination with vaccinia virus, e.g., within about 60 seconds, about two minutes, about 5 minutes, about 15 minutes, about 60 minutes, about two hours, or about 4 hours of administering a vaccinia virus vaccine.

In some embodiments, the present invention provides methods for reducing the risk that an individual will develop a pathological poxvirus infection, e.g., smallpox virus infection or vaccinia virus infection, in an individual who has not been vaccinated with vaccinia virus, or individuals who were previously vaccinated but in whom protective immunity has lapsed. Individuals who were previously vaccinated with vaccinia virus and who lack protective immunity to smallpox virus include individuals who were immunized more than about three years prior to treatment with a subject method. Individuals to be treated include individuals who are expected to be exposed to a poxvirus or who are at risk of being exposed to a poxvirus, but who have not yet been infected. Such individuals include, but are not limited to, military personnel and health care professionals. In some of these embodiments, the methods involve administering an effective amount of a Type I or a Type III interferon receptor agonist. In other embodiments, the methods involve administering an effective amount of a Type II interferon receptor agonist. In other embodiments, the methods involve administering effective amounts of a Type I or a Type III interferon receptor agonist in combination therapy with an effective amount of a Type II interferon receptor agonist. In some embodiments, an interferon treatment is accompanied by vaccinia virus vaccine. In some embodiments, interferon therapy is begun from one day to about 14 days, or from about one day to about 7 days before vaccination with vaccinia virus. In other embodiments, interferon treatment begins on the same day as vaccination with vaccinia virus, e.g., within about 60 seconds, about two minutes, about 5 minutes, about 15 minutes, about 60 minutes, about two hours, or about 4 hours of administering a vaccinia virus vaccine.

In some embodiments, the present invention provides methods of reducing viral load, and/or reducing the time to viral clearance, and/or reducing morbidity or mortality in an individual who has not been vaccinated with vaccinia virus vaccine, or who was previously vaccinated with vaccinia virus but in whom protective immunity has lapsed, and who has been exposed to a poxvirus, e.g., smallpox virus. In some of these embodiments, the methods involve administering an effective amount of a Type I or a Type III interferon receptor agonist within 24 hours of exposure to the poxvirus. In other embodiments, the methods involve administering an effective amount of a Type II interferon receptor agonist within 24 hours of exposure to the poxvirus. In other embodiments, the methods involve administering effective amounts of a Type I or a Type III interferon receptor agonist in combination therapy with an effective amount of a Type II interferon receptor agonist within 24 hours of exposure to the poxvirus. In some embodiments, an interferon treatment is accompanied by vaccinia virus vaccine. In some embodiments, interferon treatment begins on the same day as vaccination with vaccinia virus, e.g., within about 60 seconds, about two minutes, about 5 minutes, about 15 minutes, about 60 minutes, about two hours, or about 4 hours of administering a vaccinia virus vaccine.

In some embodiments, the present invention provides methods of reducing viral load, and/or reducing the time to viral clearance, and/or reducing morbidity or mortality in an individual who has been vaccinated with vaccinia virus (e.g., an individual who has been vaccinated with vaccinia virus within the last month, within the last two months, within the last 6 months, within the last year, within the last two years, or within the last three years prior to treatment with a subject method), and who has been exposed to a poxvirus, e.g., smallpox virus. In some of these embodiments, the methods involve administering an effective amount of a Type I or a Type II interferon receptor agonist within 24 hours of exposure to the poxvirus. In other embodiments, the methods involve administering an effective amount of a Type II interferon receptor agonist within 24 hours of exposure to the poxvirus. In other embodiments, the methods involve administering effective amounts of a Type I or a Type III interferon receptor agonist in combination therapy with an effective amount of a Type II interferon receptor agonist within 24 hours of exposure to the poxvirus.

In some embodiments, the present invention provides methods of reducing viral load, and/or reducing the time to viral clearance, and/or reducing morbidity or mortality in an individual who has not been vaccinated with vaccinia virus vaccine, or who was previously vaccinated with vaccinia virus but in whom protective immunity has lapsed, and who has been exposed to a poxvirus, e.g., smallpox virus. In some of these embodiments, the methods involve administering an effective amount of a Type I or a Type II interferon receptor agonist within 48 hours of exposure to the poxvirus. In other embodiments, the methods involve administering an effective amount of a Type II interferon receptor agonist within 48 hours of exposure to the poxvirus. In other embodiments, the methods involve administering effective amounts of a Type I or a Type III interferon receptor agonist in combination therapy with an effective amount of a Type II interferon receptor agonist within 48 hours of exposure to the poxvirus. In some embodiments, an interferon treatment is accompanied by vaccinia virus vaccine. In some embodiments, interferon treatment begins on the same day as vaccination with vaccinia virus, e.g., within about 60 seconds, about two minutes, about 5 minutes, about 15 minutes, about 60 minutes, about two hours, or about 4 hours of administering a vaccinia virus vaccine.

In some embodiments, the present invention provides methods of reducing viral load, and/or reducing the time to viral clearance, and/or reducing morbidity or mortality in an individual who has been vaccinated with vaccinia virus (e.g., an individual who has been vaccinated with vaccinia virus within the last month, within the last two months, within the last 6 months, within the last year, within the last two years, or within the last three years prior to treatment with a subject method), and who has been exposed to a poxvirus, e.g., smallpox virus. In some of these embodiments, the methods involve administering an effective amount of a Type I or a Type II interferon receptor agonist within 48 hours of exposure to the poxvirus. In other embodiments, the methods involve administering an effective amount of a Type II interferon receptor agonist within 48 hours of exposure to the poxvirus. In other embodiments, the methods involve administering effective amounts of a Type I or a Type III interferon receptor agonist in combination therapy with an effective amount of a Type II interferon receptor agonist within 48 hours of exposure to the poxvirus.

In some embodiments, the present invention provides methods of reducing viral load, and/or reducing the time to viral clearance, and/or reducing morbidity or mortality in an individual who has not been vaccinated with vaccinia virus vaccine, or who was previously vaccinated with vaccinia virus but in whom protective immunity has lapsed, and who has been exposed to a poxvirus, e.g., smallpox virus. The methods involve administering an interferon treatment more than 48 hours after exposure to the poxvirus, e.g., from 72 hours to about 35 days, e.g., 72 hours, 4 days, 5 days, 6 days, or 7 days after exposure, or from about 7 days to about 10 days, from about 10 days to about 14 days, from about 14 days to about 17 days, from about 17 days to about 21 days, from about 21 days to about 25 days, from about 25 days to about 30 days, or from about 30 days to about 35 days after exposure to the poxvirus. In some of these embodiments, the methods involve administering an effective amount of a Type I or a Type III interferon receptor agonist more than 48 hours after exposure to the poxvirus. In other embodiments, the methods involve administering an effective amount of a Type II interferon receptor agonist more than 48 hours after exposure to the poxvirus. In other embodiments, the methods involve administering effective amounts of a Type I or a Type III interferon receptor agonist in combination therapy with an effective amount of a Type II interferon receptor agonist more than 48 hours after exposure to the poxvirus. In some embodiments, an interferon treatment is accompanied by vaccinia virus vaccine. In some embodiments, interferon treatment begins on the same day as vaccination with vaccinia virus, e.g., within about 60 seconds, about two minutes, about 5 minutes, about 15 minutes, about 60 minutes, about two hours, or about 4 hours of administering a vaccinia virus vaccine.

In some embodiments, the present invention provides methods of reducing viral load, and/or reducing the time to viral clearance, and/or reducing morbidity or mortality in an individual who has been vaccinated with vaccinia virus (e.g., an individual who has been vaccinated with vaccinia virus within the last month, within the last two months, within the last 6 months, within the last year, within the last two years, or within the last three years prior to treatment with a subject method), and who has been exposed to a poxvirus, e.g., smallpox virus. The methods involve administering an interferon treatment more than 48 hours after exposure to the poxvirus, e.g., from 72 hours to about 35 days, e.g., 72 hours, 4 days, 5 days, 6 days, or 7 days after exposure, or from about 7 days to about 10 days, from about 10 days to about 14 days, from about 14 days to about 17 days, from about 17 days to about 21 days, from about 21 days to about 25 days, from about 25 days to about 30 days, or from about 30 days to about 35 days after exposure to the poxvirus. In some of these embodiments, the methods involve administering an effective amount of a Type I or a Type III interferon receptor agonist more than 48 hours after exposure to the poxvirus. In other embodiments, the methods involve administering an effective amount of a Type II interferon receptor agonist more than 48 hours after exposure to the poxvirus. In other embodiments, the methods involve administering effective amounts of a Type I or a Type III interferon receptor agonist in combination therapy with an effective amount of a Type II interferon receptor agonist more than 48 hours after exposure to the poxvirus.

In some embodiments, the present invention provides any of the above-described methods in which Type I interferon receptor agonist therapy is employed and in which the Type I interferon receptor agonist is an IFN-α or a PEGylated IFN-α conjugate (PEG-IFN-α). In some of these embodiments, the IFN-α is a consensus interferon (CIFN) or a PEGylated consensus interferon conjugate (PEG-CIFN).

In some embodiments, the present invention provides any of the above-described methods in which Type II interferon receptor agonist therapy is employed and in which the Type II interferon receptor agonist is an IFN-γ.

Type I Interferon Receptor Agonists

In any of the above-described methods, in some embodiments a Type I interferon receptor agonist is administered. Type I interferon receptor agonists include an IFN-α; an IFN-β; an IFN-tau; an IFN-ω; antibody agonists specific for a Type I interferon receptor; and any other agonist of Type I interferon receptor, including non-polypeptide agonists.

Interferon-Alpha

Any known IFN-α can be used in the instant invention. The term “interferon-alpha” as used herein refers to a family of related polypeptides that inhibit viral replication and cellular proliferation and modulate immune response. The term “IFN-α ” includes naturally occurring IFN-α; synthetic IFN-α; derivatized IFN-α (e.g., PEGylated IFN-α, glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Weilcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con₁ is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods. Use of CIFN is of particular interest.

Also suitable for use in the present invention are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present invention are gene-shuffled forms of IFN-α. See., e.g., Masci et al. (2003) Curr. Oncol. Rep. 5:108-113.

PEGylated Interferon-Alpha

The term “IFN-α ” also encompasses derivatives of IFN-α that are derivatized (e.g., are chemically modified) to alter certain properties such as serum half-life. As such, the term “IFN-α ” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

Any of the above-mentioned IFN-α polypeptides can be modified with one or more polyethylene glycol moieties, i.e., PEGylated. The PEG molecule of a PEGylated IFN-α polypeptide is conjugated to one or more amino acid side chains of the IFN-α polypeptide. In some embodiments, the PEGylated IFN-α contains a PEG moiety on only one amino acid. In other embodiments, the PEGylated IFN-α contains a PEG moiety on two or more amino acids, e.g., the IFN-α contains a PEG moiety attached to two, three, four, five, six, seven, eight, nine, or ten different amino acid residues.

IFN-α may be coupled directly to PEG (i.e., without a linking group) through an amino group, a sulfhydryl group, a hydroxyl group, or a carboxyl group.

In some embodiments, the PEGylated IFN-α is PEGylated at or near the amino terminus (N-terminus) of the IFN-α polypeptide, e.g., the PEG moiety is conjugated to the IFN-α polypeptide at one or more amino acid residues from amino acid 1 through amino acid 4, or from amino acid 5 through about 10.

In other embodiments, the PEGylated IFN-α is PEGylated at one or more amino acid residues from about 10 to about 28.

In other embodiments, the PEGylated IFN-α is PEGylated at or near the carboxyl terminus (C-terminus) of the IFN-α polypeptide, e.g., at one or more residues from amino acids 156-166, or from amino acids 150 to 155.

In other embodiments, the PEGylated IFN-α is PEGylated at one or more amino acid residues at one or more residues from amino acids 100-114.

The polyethylene glycol derivatization of amino acid residues at or near the receptor-binding and/or active site domains of the IFN-α protein can disrupt the functioning of these domains. In certain embodiments of the invention, amino acids at which PEGylation is to be avoided include amino acid residues from amino acid 30 to amino acid 40; and amino acid residues from amino acid 113 to amino acid 149.

In some embodiments, PEG is attached to IFN-α via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine.

Methods for malting succinimidyl propionate (SPA) and succinimidyl butanoate (SBA) ester-activated PEGs are described in U.S. Pat. No. 5,672,662 (Harris, et al.) and WO 97/03106.

Methods for attaching a PEG to an IFN-α polypeptide are known in the art, and any known method can be used. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); U.S. Pat. No. 5,985,265; U.S. Pat. No. 5,672,662 (Harris, et al.) and WO 97/03106.

Pegylated IFN-α, and methods for making same, is discussed in, e.g., U.S. Pat. Nos. 5,382,657; 5,981,709; 5,985,265; and 5,951,974. Pegylated IFN-α encompasses conjugates of PEG and any of the above-described IFN-α molecules, including, but not limited to, PEG conjugated to interferon alpha-2a (Roferon, Hoffman LaRoche, Nutley, N.J.), where PEGylated Roferon is known as Pegasys (Hoffman LaRoche); interferon alpha 2b (Intron, Schering-Plough, Madison, N.J.), where PEGylated Intron is known as PEG-Intron (Schering-Plough); interferon alpha-2c (Berofor Alpha, Boehringer Ingelheim, Ingelheim, Germany); and consensus interferon (CIFN) as defined by determination of a consensus sequence of naturally occurring interferon alphas (Infergen®, InterMune, Inc., Brisbane, Calif.), where PEGylated Infergen is referred to as PEG-Infergen.

In many embodiments, the PEG is a monomethoxyPEG molecule that reacts with primary amine groups on the IFN-α polypeptide. Methods of modifying polypeptides with monomethoxy PEG via reductive alkylation are known in the art. See, e.g., Chamow et al. (1994) Bioconj. Chem. 5:133-140.

In one non-limiting example, PEG is linked to IFN-α via an SPA linking group. SPA esters of PEG, and methods for making same, are described in U.S. Pat. No. 5,672,662. SPA linkages provide for linkage to free amine groups on the IFN-α polypeptide.

For example, a PEG molecule is covalently attached via a linkage that comprises an amide bond between a propionyl group of the PEG moiety and the epsilon amino group of a surface-exposed lysine residue in the IFN-α polypeptide. Such a bond can be formed, e.g., by condensation of an α-methoxy, omega propanoic acid activated ester of PEG (mPEGspa).

As one non-limiting example, one monopegylated CIFN conjugate preferred for use herein has a linear PEG moiety of about 30 kD attached via a covalent linkage to the CIFN polypeptide, where the covalent linkage is an amide bond between a propionyl group of the PEG moiety and the epsilon amino group of a surface-exposed lysine residue in the CIFN polypeptide, where the surface-exposed lysine residue is chosen from lys³¹, lys⁵⁰, lys⁷¹, lys⁸⁴, lys¹²¹, lys¹²², lys¹³⁴, lys¹³⁵, and lys¹⁶⁵, and the amide bond is formed by condensation of an α-methoxy, omega propanoic acid activated ester of PEG.

Polyethylene Glycol

Polyethylene glycol suitable for conjugation to an IFN-α polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine.

In other embodiments, PEG is derivatized so that it is reactive with free carboxyl groups in the IFN-α polypeptide, e.g., the free carboxyl group at the carboxyl terminus of the IFN-α polypeptide. Suitable derivatives of PEG that are reactive with the free carboxyl group at the carboxyl-terminus of IFN-α include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues to react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

The PEG can be conjugated directly to the IFN-α polypeptide, or through a linker. In some embodiments, a linker is added to the IFN-α polypeptide, forming a linker-modified IFN-α polypeptide. Such linkers provide various functionalities, e.g., reactive groups such sulfhydryl, amino, or carboxyl groups to couple a PEG reagent to the linker-modified IFN-α polypeptide.

In some embodiments, the PEG conjugated to the IFN-α polypeptide is linear. In other embodiments, the PEG conjugated to the IFN-α polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to IFN-α has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

Preparing PEG-IFN-α Conjugates

As discussed above, the PEG moiety can be attached, directly or via a linker, to an amino acid residue at or near the N-terminus, internally, or at or near the C-terminus of the IFN-α polypeptide. Conjugation can be carried out in solution or in the solid phase.

N-Terminal Linkage

Methods for attaching a PEG moiety to an amino acid residue at or near the N-terminus of an IFN-α polypeptide are known in the art. See, e.g., U.S. Pat. No. 5,985,265.

In some embodiments, known methods for selectively obtaining an N-terminally chemically modified IFN-α are used. For example, a method of protein modification by reductive alkylation which exploits differential reactivity of different types of primary amino groups (lysine versus the N-terminus) available for derivatization in a particular protein can be used. Under the appropriate reaction conditions, substantially selective derivatization of the protein at the N-terminus with a carbonyl group containing polymer is achieved. The reaction is performed at pH which allows one to take advantage of the pKₐ differences between the ε-amino groups of the lysine residues and that of the α-amino group of the N-terminal residue of the protein. By such selective derivatization attachment of a PEG moiety to the IFN-α is controlled: the conjugation with the polymer takes place predominantly at the N-terminus of the IFN-α and no significant modification of other reactive groups, such as the lysine side chain amino groups, occurs.

C-Terminal Linkage

N-terminal-specific coupling procedures such as described in U.S. Pat. No. 5,985,265 provide predominantly monoPEGylated products. However, the purification procedures aimed at removing the excess reagents and minor multiply PEGylated products remove the N-terminal blocked polypeptides. In terms of therapy, such processes lead to significant increases in manufacturing costs. For example, examination of the structure of the well-characterized Infergen® Alfacon-1 CIFN polypeptide amino acid sequence reveals that the clipping is approximate 5% at the carboxyl terminus and thus there is only one major C-terminal sequence. Thus, in some embodiments, N-terminally PEGylated IFN-α is not used; instead, the IFN-α polypeptide is C-terminally PEGylated.

An effective synthetic as well as therapeutic approach to obtain mono PEGylated Infergen product is therefore envisioned as follows:

A PEG reagent that is selective for the C-terminal can be prepared with or without spacers. For example, polyethylene glycol modified as methyl ether at one end and having an amino function at the other end may be used as the starting material.

Preparing or obtaining a water-soluble carbodiimide as the condensing agent can be carried out. Coupling IFN-α (e.g., Infergen® Alfacon-1 CIFN or consensus interferon) with a water-soluble carbodiimide as the condensing reagent is generally carried out in aqueous medium with a suitable buffer system at an optimal pH to effect the amide linkage. A high molecular weight PEG can be added to the protein covalently to increase the molecular weight

The reagents selected will depend on process optimization studies. A non-limiting example of a suitable reagent is EDAC or 1-ethyl-3-(3-dimethylaminopropyl) carbodiimide. The water solubility of EDAC allows for direct addition to a reaction without the need for prior organic solvent dissolution. Excess reagent and the isourea formed as the by-product of the cross-linking reaction are both water-soluble and may easily be removed by dialysis or gel filtration. A concentrated solution of EDAC in water is prepared to facilitate the addition of a small molar amount to the reaction. The stock solution is prepared and used immediately in view of the water labile nature of the reagent. Most of the synthetic protocols in literature suggest the optimal reaction medium to be in pH range between 4.7 and 6.0. However the condensation reactions do proceed without significant losses in yields up to pH 7.5. Water may be used as solvent. In view of the contemplated use of Infergen, preferably the medium will be 2-(N-morpholino)ethane sulfonic acid buffer pre-titrated to pH between 4.7 and 6.0. However, 0.1M phosphate in the pH 7-7.5 may also be used in view of the fact that the product is in the same buffer. The ratios of PEG amine to the IFN-α molecule is optimized such that the C-terminal carboxyl residue(s) are selectively PEGylated to yield monoPEGylated derivative(s).

Even though the use of PEG amine has been mentioned above by name or structure, such derivatives are meant to be exemplary only, and other groups such as hydrazine derivatives as in PEG-NH—NH₂ which will also condense with the carboxyl group of the IFN-α protein, can also be used. In addition to aqueous phase, the reactions can also be conducted on solid phase. Polyethylene glycol can be selected from list of compounds of molecular weight ranging from 300-40000. The choice of the various polyethylene glycols will also be dictated by the coupling efficiency and the biological performance of the purified derivative in vitro and in vivo i.e., circulation times, anti viral activities etc.

Additionally, suitable spacers can be added to the C-terminal of the protein. The spacers may have reactive groups such as SH, NH₂ or COOH to couple with appropriate PEG reagent to provide the high molecular weight. IFN-α derivatives. A combined solid/solution phase methodology can be devised for the preparation of C-terminal pegylated interferons. For example, the C-terminus of IFN-α is extended on a solid phase using a Gly-Gly-Cys-NH₂ spacer and then monopegylated in solution using activated dithiopyridyl-PEG reagent of appropriate molecular weights. Since the coupling at the C-terminus is independent of the blocking at the N-terminus, the envisioned processes and products will be beneficial with respect to cost (a third of the protein is not wasted as in N-terminal PEGylation methods) and contribute to the economy of the therapy to treat poxvirus infection.

There may be a more reactive carboxyl group of amino acid residues elsewhere in the molecule to react with the PEG reagent and lead to monoPEGylation at that site or lead to multiple PEGylations in addition to the —COOH group at the C-terminus of the IFN-α. It is envisioned that these reactions will be minimal at best owing to the steric freedom at the C-terminal end of the molecule and the steric hindrance imposed by the carbodiimides and the PEG reagents such as in branched chain molecules. It is therefore the preferred mode of PEG modification for Infergen and similar such proteins, native or expressed in a host system, which may have blocked N-termini to varying degrees to improve efficiencies and maintain higher in vivo biological activity.

Another method of achieving C-terminal PEGylation is as follows. Selectivity of C-terminal PEGylation is achieved with a sterically hindered reagent which excludes reactions at carboxyl residues either buried in the helices or internally in IFN-α. For example, one such reagent could be a branched chain PEG ˜40 kd in molecular weight and this agent could be synthesized as follows:

OH₃C—(CH₂CH₂O)ₙ—CH₂CH₂NH₂+Glutamic Acid i.e., HOCO—CH₂CH₂CH(NH2)-COOH is condensed with a suitable agent e.g., dicyclohexyl carbodiimide or water-soluble EDC to provide the branched chain PEG agent OH₃C—(CH₂CH₂O)ₙ—CH₂CH₂NHCOCH(NH₂)CH₂OCH₃—(CH₂CH₂O)ₙ—CH₂CH₂NHCOCH₂.

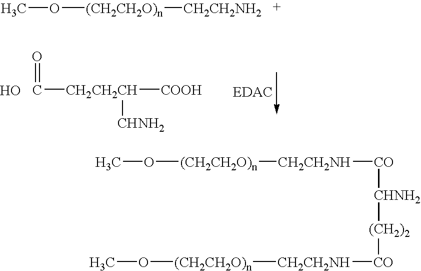

This reagent can be used in excess to couple the amino group with the free and flexible carboxyl group of IFN-α to form the peptide bond.

If desired, PEGylated IFN-α is separated from unPEGylated IFN-α using any known method, including, but not limited to, ion exchange chromatography, size exclusion chromatography, and combinations thereof. For example, where the PEG-IFN-α conjugate is a monoPEGylated IFN-α, the products are first separated by ion exchange chromatography to obtain material having a charge characteristic of monoPEGylated material (other multi-PEGylated material having the same apparent charge may be present), and then the monoPEGylated materials are separated using size exclusion chromatography.

IFN-β

The term interferon-beta (“IFN-β”) includes IFN-β polypeptides that are naturally occurring; non-naturally-occurring IFN-β polypeptides; and analogs of naturally occurring or non-naturally occurring IFN-β that retain antiviral activity of a parent naturally-occurring or non-naturally occurring IFN-β.

Any of a variety of beta interferons can be delivered by the continuous delivery method of the present invention. Suitable beta interferons include, but are not limited to, naturally-occurring IFN-β; IFN-β1a, e.g., Avonex® (Biogen, Inc.), and Rebif® (Serono, SA); IFN-β1b (Betaseron®; Berlex); and the like.

The IFN-β formulation may comprise an N-blocked species, wherein the N-terminal amino acid is acylated with an acyl group, such as a formyl group, an acetyl group, a malonyl group, and the like. Also suitable for use is a consensus IFN-β.

IFN-β polypeptides can be produced by any known method. DNA sequences encoding IFN-β may be synthesized using standard methods. In many embodiments, IFN-β polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-β is “recombinant IFN-β.” Where the host cell is a bacterial host cell, the IFN-β is modified to comprise an N-terminal methionine.

It is to be understood that IFN-β as described herein may comprise one or more modified amino acid residues, e.g., glycosylations, chemical modifications, and the like.

IFN-tau

The term interferon-tau includes IFN-tau polypeptides that are naturally occurring; non-naturally-occurring IFN-tau polypeptides; and analogs of naturally occurring or non-naturally occurring IFN-tau that retain antiviral activity of a parent naturally-occurring or non-naturally occurring IFN-tau.

Suitable tau interferons include, but are not limited to, naturally-occurring IFN-tau; Tauferon® (Pepgen Corp.); and the like.

IFN-tau may comprise an amino acid sequence as set forth in any one of GenBank Accession Nos. P15696; P56828; P56832; P56829; P56831; Q29429; Q28595; Q28594; S08072; Q08071; Q08070; Q08053; P56830; P28169; P28172; and P28171. The sequence of any known IFN-tau polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

The IFN-tau formulation may comprise an N-blocked species, wherein the N-terminal amino acid is acylated with an acyl group, such as a formyl group, an acetyl group, a malonyl group, and the like. Also suitable for use is a consensus IFN-tau.

IFN-tau polypeptides can be produced by any known method. DNA sequences encoding IFN-tau may be synthesized using standard methods. In many embodiments, IFN-tau polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-tau is “recombinant IFN-tau.” Where the host cell is a bacterial host cell, the IFN-tau is modified to comprise an N-terminal methionine.

It is to be understood that IFN-tau as described herein may comprise one or more modified amino acid residues, e.g., glycosylations, chemical modifications, and the like.

IFN-ω

The term interferon-omega (“IFN-ω”) includes IFN-ω polypeptides that are naturally occurring; non-naturally-occurring IFN-ω polypeptides; and analogs of naturally occurring or non-naturally occurring IFN-ω that retain antiviral activity of a parent naturally-occurring or non-naturally occurring IFN-ω.

Any known omega interferon can be delivered by the continuous delivery method of the present invention. Suitable IFN-ω include, but are not limited to, naturally-occurring IFN-ω; recombinant IFN-ω, e.g., Biomed 510 (BioMedicines); and the like.

IFN-ω may comprise an amino acid sequence as set forth in GenBank Accession No. NP_{—}002168; or AAA70091. The sequence of any known IFN-ω polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

The IFN-ω formulation may comprise an N-blocked species, wherein the N-terminal amino acid is acylated with an acyl group, such as a formyl group, an acetyl group, a malonyl group, and the like. Also suitable for use is a consensus IFN-ω.

IFN-ω polypeptides can be produced by any known method. DNA sequences encoding IFN-ωmay be synthesized using standard methods. In many embodiments, IFN-ω polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-ω is “recombinant IFN-ω.” Where the host cell is a bacterial host cell, the IFN-ωis modified to comprise an N-terminal methionine.

It is to be understood that IFN-ω as described herein may comprise one or more modified amino acid residues, e.g., glycosylations, chemical modifications, and the like.

Type III Interferon Receptor Agonists

In any of the above-described methods or apparatus, the interferon receptor agonist is in some embodiments an agonist of a Type III interferon receptor (e.g., “a Type III interferon agonist”). Type III interferon agonists include an IL-28b polypeptide; and IL-28a polypeptide; and IL-29 polypeptide; antibody specific for a Type III interferon receptor; and any other agonist of Type III interferon receptor, including non-polypeptide agonists.

IL-28A, IL-28B, and IL-29 (referred to herein collectively as “Type III interferons” or “Type III IFNs”) are described in Sheppard et al. (2003) Nature 4:63-68. Each polypeptide binds a heterodimeric receptor consisting of IL-10 receptor β chain and an IL-28 receptor α. Sheppard et al. (2003), supra. The amino acid sequences of IL-28A, IL-28B, and IL-29 are found under GenBank Accession Nos. NP_{—}742150, NP-742151, and NP_{—}742152, respectively.

The amino acid sequence of a Type III IFN polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation; such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability. The polypeptides may be fused to albumin.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for lining to other molecules or to a surface. Thus cysteines can be used to make thioethers, histidines for linking to a metal ion complex, carboxyl groups for forming amides or esters, amino groups for forming amides, and the like.

Type II Interferon Receptor Agonists

Type II interferon receptor agonists include any naturally-occurring or non-naturally-occurring ligand of a human Type II interferon receptor which binds to and causes signal transduction via the receptor. Type II interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

A specific example of a Type II interferon receptor agonist is IFN-γ and variants thereof. While the present invention exemplifies use of an IFN-γ polypeptide, it will be readily apparent that any Type II interferon receptor agonist can be used in a subject method.

Interferon-Gamma

The nucleic acid sequences encoding IFN-γ polypeptides may be accessed from public databases, e.g., Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

IFN-γ1b (Actimmune®; human interferon) is a single-chain polypeptide of 140 amino acids. It is made recombinantly in E. coli and is unglycosylated. Rinderknecht et al. (1984) J. Biol. Chem. 259:6790-6797. Recombinant IFN-γ as discussed in U.S. Pat. No. 6,497,871 is also suitable for use herein.

The IFN-γ to be used in the methods of the present invention may be any of natural IFN-γs, recombinant IFN-γs and the derivatives thereof so far as they have an IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra, consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG that, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxyl terminus relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e., will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In one embodiment, the invention contemplates the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001. Also included are modifications of glycosylation, e.g., those made by modifying the glycosylation patterns of a polypeptide during its synthesis and processing or in further processing steps; e.g., by exposing the polypeptide to enzymes that affect glycosylation, such as mammalian glycosylating or deglycosylating enzymes. Also embraced are sequences that have phosphorylated amino acid residues, e.g., phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g., D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight; and for therapeutic purposes, usually at least about 99.5% by weight, in relation to contaminants related to the method of preparation of the product and its purification. Usually, the percentages will be based upon total protein.

Dosages, Formulations, and Routes of Administration

A Type I or Type III interferon receptor agonist, a Type II interferon receptor agonist, or a Type I or Type III interferon receptor agonist and a Type II interferon receptor agonist are administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, the active agents may be administered to the host using any convenient means capable of resulting in the desired therapeutic effect. Thus, the agents can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, subcutaneous, intramuscular, transdermal, intratracheal, etc., administration. In some embodiments, two different routes of administration are used. In some embodiments, IFN-α is administered subcutaneously. In other embodiments, IFN-γ is administered subcutaneously. In other embodiments, both IFN-γ and IFN-α are administered subcutaneously.

Subcutaneous administration of a Type I, a Type III, or a Type II interferon receptor agonist is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of an interferon receptor agonist to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, a Type I or Type III, or a Type II interferon receptor agonist interferon receptor agonist, is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent (e.g., a Type I or Type III interferon receptor agonist, e.g., IFN-α; or a Type II interferon receptor agonist, e.g., IFN-γ) is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a Type I or Type III interferon receptor agonist or a Type II interferon receptor agonist is delivered using an implantable drug delivery system, e.g., a system that is programmable to provide for administration of the interferon receptor agonist. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonfuil, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

In some embodiments, at least one dose of a Type II interferon receptor agonist is administered concurrently with at least one dose of a Type I or Type III interferon receptor agonist. As used herein, the term “concurrently” indicates that the Type II interferon receptor agonist and the Type I or Type III interferon receptor agonist are administered separately and are administered within about 5 seconds to about 15 seconds, within about 15 seconds to about 30 seconds, within about 30 seconds to about 60 seconds, within about 1 minute to about 5 minutes, within about 5 minutes to about 15 minutes, within about 15 minutes to about 30 minutes, within about 30 minutes to about 60 minutes, within about 1 hour to about 2 hours, within about 2 hours to about 6 hours, within about 6 hours to about 12 hours, within about 12 hours to about 24 hours, or within about 24 hours to about 48-hours of one another.

In some embodiments, a Type II interferon receptor agonist is administered during the entire course of Type I or Type III interferon receptor agonist treatment. In other embodiments, a Type II interferon receptor agonist is administered for a period of time that is overlapping with that of the Type I or Type II interferon receptor agonist treatment, e.g., the a Type II interferon receptor agonist treatment can begin before the Type I or Type III interferon receptor agonist treatment begins and end before the Type I or Type III interferon receptor agonist treatment ends; the a Type II interferon receptor agonist treatment can begin after the Type I or Type III interferon receptor agonist treatment begins and end after the a Type II interferon receptor agonist treatment ends; the a Type II interferon receptor agonist treatment can begin after the Type I or Type III interferon receptor agonist treatment begins and end before the Type I or Type III interferon receptor agonist treatment ends; or the a Type II interferon receptor agonist γ treatment can begin before the Type I or Type III interferon receptor agonist treatment begins and end after the Type I or Type III interferon receptor agonist treatment ends.

In connection with each of the methods described herein, the invention provides embodiments in which the Type I or Type III interferon receptor agonist and/or a Type II interferon receptor agonist is administered to the patient by a controlled drug delivery device. In some embodiments, the Type I or Type III interferon receptor agonist and/or a Type II interferon receptor agonist is delivered to the patient substantially continuously or continuously by the controlled drug delivery device. Optionally, an implantable infusion pump is used to deliver the Type I or Type III interferon receptor agonist and/or a Type II interferon receptor agonist to the patient substantially continuously or continuously by subcutaneous infusion.

In other embodiments, the Type I or Type III interferon receptor agonist and/or a Type II interferon receptor agonist is administered to the patient so as to achieve and maintain a desired average daily serum concentration of the Type I or Type III interferon receptor agonist and/or a Type II interferon receptor agonist at a substantially steady state for the duration of the Type I or Type III interferon receptor agonist and/or a Type II interferon receptor agonist therapy. Optionally, an implantable infusion pump is used to deliver the Type I or Type III interferon receptor agonist and/or a Type II interferon receptor agonist to the patient by subcutaneous infusion so as to achieve and maintain a desired average daily serum concentration of the Type I or Type III interferon receptor agonist and/or a Type II interferon receptor agonist at a substantially steady state for the duration of the Type I or Type III interferon receptor agonist and/or a Type II interferon receptor agonist therapy.

A Type II interferon receptor agonist can be administered daily, twice daily, every other day, twice a week, three times a week, or substantially continuously or continuously. Effective dosages of a Type II interferon receptor agonist can range from about 1 μg to about 1000 μg.

In some embodiments, the Type II interferon receptor agonist is IFN-γ. Effective dosages of IFN-γ range from about 0.5 μg/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, or substantially continuously or continuously.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

A Type I or a Type III interferon receptor agonist can be administered twice daily, daily, every other day, once a week, twice a week, three times a week, every other week, three times per month, or once monthly, substantially continuously or continuously.

In some embodiments, the Type I interferon receptor agonist is an IFN-α. Effective dosages of an IFN-α range from about 3 μg to about 27 μg, from about 3 MU to about 10 MU, from about 90 μg to about 180 μg, or from about 18 μg to about 90 μg.

Effective dosages of Infergen® consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b range from 3 million Units (MU) to 10 MU per dose. Effective dosages of PEGASYS®PEGylated IFN-α2a contain an amount of about 90 μg to 270 μg, or about 180 μg, of drug per dose. Effective dosages of PEG-INTRON®PEGylated IFN-α2b contain an amount of about 0.5 μg to 3.0 μg of drug per kg of body weight per dose. Effective dosages of PEGylated consensus interferon (PEG-CIFN) contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN contain an amount of about 45 μg to about 270 μg, or about 60 μg to about 180 μg, or about 90 μg to about 120 μg, of drug per dose. IFN-α can be administered daily, every other day, once a week, three times a week, every other week, three times per month, once monthly, substantially continuously or continuously.

In some embodiments, a Type I or a Type III interferon receptor agonist is administered in a first dosing regimen, followed by a second dosing regimen. The first dosing regimen of Type I or a Type III interferon receptor agonist (also referred to as “the induction regimen”) generally involves administration of a higher dosage of the Type I or Type III interferon receptor agonist. For example, in the case of Infergen® consensus IFN-α (CIFN), the first dosing regimen comprises administering CIFN at about 9 μg, about 15 μg, about 18 μg, or about 27 μg. The first dosing regimen can encompass a single dosing event, or at least two or more dosing events. The first dosing regimen of the Type I or Type III interferon receptor agonist can be administered daily, every other day, three times a week, every other week, three times per month, once monthly, substantially continuously or continuously.

The first dosing regimen of the Type I or Type III interferon receptor agonist is administered for a first period of time, which time period can be at least about 4 weeks, at least about 8 weeks, or at least about 12 weeks.

The second dosing regimen of the Type I or Type III interferon receptor agonist (also referred to as “the maintenance dose”) generally involves administration of a lower amount of the Type I or Type III interferon receptor agonist. For example, in the case of CIFN, the second dosing regimen comprises administering CIFN at a dose of at least about 3 μg, at least about 9 μg, at least about 15 μg, or at least about 18 μg. The second dosing regimen can encompass a single dosing event, or at least two or more dosing events.

The second dosing regimen of the Type I or Type III interferon receptor agonist can be administered daily, every other day, three times a week, every other week, three times per month, once monthly, substantially continuously or continuously.

In some embodiments, where an “induction”/“maintenance” dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase. In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, the Type II interferon receptor agonist treatment is discontinued once Type I or a Type III interferon receptor agonist treatment begins.

In other embodiments, the Type I or Type III interferon receptor agonist is administered in single dosing regimen. For example, in the case of CIFN, the dose of CIFN is generally in a range of from about 3 μg to about 15 μg, or from about 9 μg to about 15 μg. The dose of Type I or a Type III interferon receptor agonist is generally administered daily, every other day, three times a week, every other week, three times per month, once monthly, or substantially continuously. The dose of the Type I or Type III interferon receptor agonist is administered for a period of time, which period can be, for example, from at least about 24 weeks to at least about 48 weeks, or longer.

In some embodiments, where a single dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase. In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with the Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, Type II interferon receptor agonist treatment is discontinued once Type I or a Type III interferon receptor agonist treatment begins.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Those of skill in the art will readily appreciate that dose levels can vary as a function of the specific compounds, the severity of the symptoms and the susceptibility of the subject to side effects. Preferred dosages for a given compound are readily determinable by those of skill in the art by a variety of means. A preferred means is to measure the physiological potency of a given compound.

In some embodiments, the Type I or Type III interferon receptor agonist and a Type II interferon receptor agonist are administered in the same formulation, and are administered simultaneously. In other embodiments, the Type I or Type III interferon receptor agonist and a Type II interferon receptor agonist are administered separately, e.g., in separate formulations. In some of these embodiments, the Type I or Type III interferon receptor agonist and a Type II interferon receptor agonist are administered separately, and are administered simultaneously. In other embodiments, the Type I or Type III interferon receptor agonist and a Type II interferon receptor agonist are administered separately and are administered within about 5 seconds to about 15 seconds, within about 15 seconds to about 30 seconds, within about 30 seconds to about 60 seconds, within about 1 minute to about 5 minutes, within about 5 minutes to about 15 minutes, within about 15 minutes to about 30 minutes, within about 30 minutes to about 60 minutes, within about 1 hour to about 2 hours, within about 2 hours to about 6 hours, within about 6 hours to about 12 hours, within about 12 hours to about 24 hours, or within about 24 hours to about 48 hours of one another.

Multiple doses of Type I or a Type III interferon receptor agonist and a Type II interferon receptor agonist can be administered, e.g., the Type I or Type III interferon receptor agonist and a Type II interferon receptor agonist can be administered once per month, twice per month, three times per month, once per week, twice per week, three times per week, four times per week, five times per week, six times per week, or daily, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, IFN-α and IFN-γ are administered in combination therapy. In some of these embodiments, the IFN-α and IFN-γ are co-formulated in a single liquid formulation that is contained in a single reservoir, for use in a drug delivery device. Thus, the present invention provides a pharmaceutical formulation comprising a single dose of IFN-α and a single dose of IFN-γ sufficient for use in any method described herein that employs the co-administration of IFN-α and IFN-γ in the treatment of a patient. In some aspects, the present invention provides a drug reservoir or other container containing IFN-α and IFN-γ co-formulated in a liquid, wherein both IFN-α and IFN-γ are present in the formulation in an amount suitable for one dose each. Dosage amounts are described herein. The reservoir can be provided in any of a variety of forms, including, but not limited to, a cartridge, a syringe, a reservoir of a continuous delivery device, and the like. The invention further provides a drug delivery device comprising (e.g., pre-loaded with) a reservoir containing a liquid formulation that comprises a single dose of IFN-α and a single dose of IFN-γ. Exemplary, non-limiting drug delivery devices include injections devices, such as pen injectors, needle/syringe devices, continuous delivery devices, and the like. Any of the dosage amounts, including synergistically effective amounts, described herein can be used in the pharmaceutical formulation, in the reservoir, or in the drug delivery device.

In other embodiments, where IFN-α and IFN-γ are administered in combination therapy, the IFN-α and IFN-γ are in separate pharmaceutical formulations contained in separate reservoirs in the same drug delivery device. The invention further provides a drug delivery device that is pre-loaded with separate reservoirs, one reservoir containing a liquid formulation comprising a single dose of IFN-α, and a second reservoir containing a liquid formulation comprising a single dose of IFN-γ. Any of the dosage amounts, including synergistically effective amounts, described herein can be used in the pharmaceutical formulations, the reservoirs, or in the drug delivery device.

In some embodiments, in a treatment method described herein, the subject method comprises administering to the patient an effective amount of a Type I interferon receptor agonist that is an IFN-α, and the subject method further comprises co-administering to the patient an effective amount of IFN-γ for the duration of the IFN-α therapy. In one embodiment, the IFN-γ is administered to the patient by bolus injection. In another embodiment, the IFN-α and IFN-γ are administered to the patient by a drug delivery device. Optionally, the device is used to deliver the IFN-α to the patient by substantially continuous or continuous administration and used to deliver the IFN-γ to the patient by bolus administration tiw, biw, qod, or qd. Optionally, the device is used to deliver the IFN-α and IFN-γ to the patient in the same manner and pattern of administration, such as substantially continuous or continuous administration. Optionally, the IFN-α and IFN-γ are contained in separate reservoirs in the drug delivery device. Optionally, the IFN-α and IFN-γ are co-formulated in a single liquid formulation that is contained in a single reservoir in the drug delivery device.

Where the agent is a polypeptide, polynucleotide (e.g., a polynucleotide encoding a Type I or a Type III interferon receptor agonist or a Type II interferon receptor agonist), it may be introduced into tissues or host cells by any number of routes, including viral infection, microinjection, or fusion of vesicles. Jet injection may also be used for intramuscular administration, as described by Furth et al. (1992) Anal. Biochem. 205:365-368. The DNA may be coated onto gold microparticles, and delivered intradermally by a particle bombardment device, or “gene gun” as described in the literature (see, for example, Tang et al. (1992) Nature 356:152-154), where gold microprojectiles are coated with the therapeutic DNA, then bombarded into skin cells.

Additional Therapeutic Agents

Any of the above-described interferon treatments can be used in conjunction with administration of an additional antiviral agents, e.g., a specific antiviral agent that is effective in treating a pathological poxvirus infection. Additional antiviral agents that are suitable for use in combination therapy include, but are not limited to, nucleotide and nucleoside analogs. Non-limiting examples include AZT (zidovudine), DDI (didanosine), DDC (dideoxycytidine), D4T (stavudine), combivir, abacavir, adefovir dipoxil, cidofovir, ribavirin, ribavirin analogs, and the like.

In some embodiments, the method further includes administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin may be administered orally in capsule or tablet form, or in the same or different administration form and in the same or different route as the interferon receptor agonist. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

Ribavirin is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day, or about 10 mg/kg body weight per day.

In some embodiments, an additional antiviral agent is administered during the entire course of interferon treatment. In other embodiments, an additional antiviral agent is administered for a period of time that is overlapping with that of the interferon treatment, e.g., the additional antiviral agent treatment can begin before the interferon treatment begins and end before the interferon treatment ends; the additional antiviral agent treatment can begin after the interferon treatment begins and end after the interferon treatment ends; the additional antiviral agent treatment can begin after the interferon treatment begins and end before the interferon treatment ends; or the additional antiviral agent treatment can begin before the interferon treatment begins and end after the interferon treatment ends.

Methods of Treatment

1. Treatment of Vaccinia Virus Infection

The present invention provides methods of treating vaccinia virus infection. The methods generally involve administering a therapeutically effective amount of a Type I or Type III interferon receptor agonist; administering a therapeutically effective amount of a Type II interferon receptor agonist; or administering therapeutically effective amounts of a Type I or Type III interferon receptor agonist and a Type II interferon receptor agonist to an individual in need thereof.

Individuals who are to be treated according to the methods of the invention include individuals who have been clinically diagnosed with a vaccinia virus infection, as well as individuals who exhibit one or more of the signs and the symptoms of clinical infection but have not yet been diagnosed with a vaccinia virus infection. Individuals who are to be treated according to the methods of the invention further include individuals who have been vaccinated with a vaccinia virus vaccine within the past 2 weeks to three years before interferon treatment; individuals who will receive vaccinia virus vaccine within 7 days to 14 days of interferon treatment; and individuals who will receive the vaccinia virus vaccine concurrently with interferon treatment. Individuals who are to be treated according to the methods of the invention further include individuals for whom vaccinia virus vaccination is contraindicated, pursuant to current guidelines from the Centers for Disease Control.

In carrying out the methods of interferon therapy for vaccinia virus infection in an individual as described above, a therapeutically effective amount of a Type I or Type III interferon receptor agonist; a therapeutically effective amount of a Type II interferon receptor agonist; or therapeutically effective amounts of a Type I or Type III interferon receptor agonist and a Type II interferon receptor agonist are administered to the individual. In some embodiments, the Type I or Type III interferon receptor agonist and the Type II interferon receptor agonist are administered in the same formulation. In other embodiments, the Type I or Type III interferon receptor agonist and the Type II interferon receptor agonist are administered in separate formulations. When administered in separate formulations, the Type I or Type III interferon receptor agonist and the Type II interferon receptor agonist can be administered substantially simultaneously, or can be administered within about 24 hours of one another. In many embodiments, the Type I or Type III interferon receptor agonist and the Type II interferon receptor agonist are administered subcutaneously in multiple doses.

A Type II interferon receptor agonist can be administered daily, twice daily, every other day, twice a week, three times a week, or substantially continuously or continuously. Effective dosages of a Type II interferon receptor agonist can range from about 1 μg to about 1000 μg. A Type II interferon receptor agonist can be administered daily, every other day, once a week, three times a week, every other week, three times per month, once monthly, substantially continuously or continuously.

In some embodiments, the Type II interferon receptor agonist is IFN-γ. Effective dosages of IFN-γ (can range from about 25 μg/dose to about 300 μg/dose, from about 10 μg/dose to about 100 μg/dose, or from about 100 μg/dose to about 1000 μg/dose.

A Type I or a Type III interferon receptor agonist can be administered daily, every other day, once a week, three times a week, every other week, three times per month, once monthly, substantially continuously or continuously.

In some embodiments, the Type I interferon receptor agonist is an IFN-α. Effective dosages of an IFN-α can range from about 1 μg to about 200 μg, e.g., from about 1 μg to about 30 μg, from about 3 μg to about 27 μg, from about 1 MU to about 20 MU, from about 3 MU to about 10 MU, from about 90 μg to about 180 μg, or from about 18 μg to about 90 μg.

Effective dosages of Infergen® consensus IFN-α can contain an amount of about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b can contain an amount of about 3 million Units (U) to about 10 MU of drug per dose. Effective dosages of PEGASYS®PEGylated IFN-α2a can contain an amount of about 90 μg to about 270 μg, or about 180 μg, of drug per dose. Effective dosages of PEG-INTRON®PEGylated IFN-α2b can contain an amount of about 0.5 μg to about 3.0 μg of drug per kg of body weight per dose. Effective dosages of PEGylated consensus interferon (PEG-CIFN) can contain an amount of about 18 μg to about 90 μg, or about 27 μg to about 60 μg, or about 45 μg, of CIFN amino acid weight per dose of PEG-CIFN. Effective dosages of monoPEG (30 kD, linear)-ylated CIFN can contain an amount of about 45 μg to about 270 μg, or about 60 μg to about 180 μg, or about 90 μg to about 120 μg, of drug per dose.

In many embodiments, the Type I or Type III interferon receptor agonist and/or the Type II interferon receptor agonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations. In some embodiments, the invention provides any of the above-described methods in which the desired dosage of IFN-α is administered subcutaneously to the patient by bolus delivery qd, qod, tiw, biw, qw, qow, three times per month, or monthly, or is administered subcutaneously to the patient per day by substantially continuous or continuous delivery, for the desired treatment duration. In other embodiments, the invention provides any of the above-described methods in which the desired dosage of PEGylated IFN-α (PEG-IFN-α) is administered subcutaneously to the patient by bolus delivery qw, qow, three times per month, or monthly for the desired treatment duration.

In some embodiments, the invention provides methods using a synergistically effective amount of a Type I or Type III interferon receptor agonist and a Type II interferon receptor agonist in the treatment of vaccinia virus infection in a patient. In some embodiments, the invention provides methods using a synergistically effective amount of an IFN-α and IFN-γ in the treatment of vaccinia virus infection in a patient. In one embodiment, the invention provides a method using a synergistically effective amount of a consensus IFN-α and IFN-γ in the treatment of vaccinia virus infection in a patient.

In general, a synergistically effective amount of a consensus interferon (CIFN) and IFN-γ suitable for use in the methods of the invention is provided by a dosage ratio of 1 μg CIFN:10 μg IFN-γ, where both CIFN and IFN-γ are unPEGylated and unglycosylated species.

In one embodiment, the invention provides a method using a synergistically effective amount of INFERGEN®consensus IFN-α and IFN-γ in the treatment of vaccinia virus infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN®consensus IPN-α and IFN-γ in the treatment of vaccinia virus infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN®consensus IFN-α and IFN-γ in the treatment of vaccinia virus infection in a patient comprising administering to the patient a dosage of INFERGEN® containing an amount of about 1 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 50 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN®consensus IFN-α and IFN-γ in the treatment of vaccinia virus infection in a patient comprising administering to the patient a dosage of INFERGEN® containing an amount of about 9 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 90 μg to about 100 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN®consensus IFN-α and IFN-γ in the treatment of vaccinia virus infection in a patient comprising administering to the patient a dosage of INFERGEN® containing an amount of about 30 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 200 μg to about 300 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of PEGylated consensus IFN-α and IFN-γ in the treatment of vaccinia virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a total weekly dosage of IFN-γ containing an amount of about 30 μg to about 1,000 μg of drug per week in divided doses administered subcutaneously qd, qod, tiw, biw, or administered substantially continuously or continuously, for the desired treatment duration.

In another embodiment, the invention provides a method using a synergistically effective amount of PEGylated consensus IFN-α and IFN-γ in the treatment of vaccinia virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a total weekly dosage of IFN-γ containing an amount of about 100 μg to about 300 μg of drug per week in divided doses administered subcutaneously qd, qod, tiw, biw, or substantially continuously or continuously, for the desired treatment duration.

In general, a synergistically effective amount of IFN-α2a or 2b or 2c and IFN-γ suitable for use in the methods of the invention is provided by a dosage ratio of 1 million Units (MU) IFN-α2a or 2b or 2c: 30 μg IFN-γ, where both IFN-α2a or 2b or 2c and IFN-γ are unPEGylated and unglycosylated species.

In another embodiment, the invention provides a method using a synergistically effective amount of IFN-α2a or 2b or 2c and IFN-γ in the treatment of vaccinia virus infection in a patient comprising administering to the patient a dosage of IFN-α2a containing an amount of about 1 MU to about 20 MU of drug per dose of IFN-α2a, 2b or 2c subcutaneously qd, qod, tiw, biw, or per day substantially continuously or continuously, in combination with a dosage of IFN-γ containing an amount of about 30 μg to about 600 μg of drug per dose of IFN-γ, subcutaneously qd, qod, tiw, biw, or per day substantially continuously or continuously, for the desired treatment duration.

In another embodiment, the invention provides a method using a synergistically effective amount of IFN-α2a or 2b or 2c and IFN-γ in the treatment of vaccinia virus infection in a patient comprising administering to the patient a dosage of IFN-α2a containing an amount of about 3 MU of drug per dose of IFN-α2a, 2b or 2c subcutaneously qd, qod, tiw, biw, or per day substantially continuously or continuously, in combination with a dosage of IFN-γ containing an amount of about 100 μg of drug per dose of IFN-γ, subcutaneously qd, qod, tiw, biw, or per day substantially continuously or continuously, for the desired treatment duration.

In another embodiment, the invention provides a method using a synergistically effective amount of IFN-α2a or 2b or 2c and IFN-γ in the treatment of vaccinia virus infection in a patient comprising administering to the patient a dosage of IFN-α2a containing an amount of about 10 MU of drug per dose of IFN-α2a, 2b or 2c subcutaneously qd, qod, tiw, biw, or per day substantially continuously or continuously, in combination with a dosage of IFN-γ containing an amount of about 300 μg of drug per dose of IFN-γ, subcutaneously qd, qod, tiw, biw, or per day substantially continuously or continuously, for the desired treatment duration.

In another embodiment, the invention provides a method using a synergistically effective amount of PEGASYS®PEGylated IFN-α2a and IFN-γ in the treatment of vaccinia virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a total weekly dosage of IFN-γ containing an amount of about 30 μg to about 1,000 μg, of drug per week administered in divided doses subcutaneously qd, qod, tiw, or biw, or administered substantially continuously or continuously, for the desired treatment duration.

In another embodiment, the invention provides a method using a synergistically effective amount of PEGASYS®PEGylated IFN-α2a and IFN-γ in the treatment of vaccinia virus infection in a patient comprising administering to the patient a dosage of PEGASYS® containing an amount of about 180 μg of drug per dose of PEGASYS®, subcutaneously qw, qow, three times per month, or monthly, in combination with a total weekly dosage of IFN-γ containing an amount of about 100 μg to about 300 μg, of drug per week administered in divided doses subcutaneously qd, qod, tiw, or biw, or administered substantially continuously or continuously, for the desired treatment duration.

In another embodiment, the invention provides a method using a synergistically effective amount of PEG-INTRON®PEGylated IFN-α2b and IFN-γ in the treatment of vaccinia virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a total weekly dosage of IFN-γ containing an amount of about 30 μg to about 1,000 μg of drug per week administered in divided doses subcutaneously qd, qod, tiw, or biw, or administered substantially continuously or continuously, for the desired treatment duration.

In another embodiment, the invention provides a method using a synergistically effective amount of PEG-INTRON®PEGylated IFN-α2b and IFN-γ in the treatment of vaccinia virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a total weekly dosage of IFN-γ containing an amount of about 100 μg to about 300 μg of drug per week administered in divided doses subcutaneously qd, qod, tiw, or biw, or administered substantially continuously or continuously, for the desired treatment duration.

The invention also provides methods for the treatment of vaccinia virus infection in which therapy with an additional antiviral agent is added to any of the Type I or Type III interferon receptor agonist and IFN-γ monotherapies or combination therapies described above.

2. Treatment of Smallpox Virus Infection

The present invention provides methods of treating smallpox virus infection by administering a therapeutically effective amount of Type I or Type III interferon receptor agonist and/or a Type II interferon receptor agonist to an individual in need thereof. Individuals who are to be treated according to the methods of the invention include individuals who have been clinically diagnosed with smallpox virus infection, as well as individuals who exhibit one or more of the signs and the symptoms of clinical infection but have not yet been diagnosed with smallpox virus infection. Individuals who are to be treated according to the methods of the invention also include individuals with anticipated exposure to smallpox virus (e.g., military personnel; health care professionals; and the like); individuals with suspected exposure to smallpox virus; and individuals with known exposure to smallpox virus.

In carrying out the methods of monotherapy for smallpox virus infection described above, a Type I or Type III interferon receptor agonist or a Type II interferon receptor agonist is administered to the individual in need of such treatment.

A Type II interferon receptor agonist can be administered daily, twice daily, every other day, twice a week, three times a week, or substantially continuously or continuously. Effective dosages of a Type II interferon receptor agonist can range from about 1 μg to about 1000 μg. A Type II interferon receptor agonist can be administered daily, every other day, once a week, three times a week, every other week, three times per month, once monthly, substantially continuously or continuously.

In some embodiments, the Type II interferon receptor agonist is IFN-γ. Effective dosages of IFN-γ can range from about 25 μg/dose to about 300 μg/dose, from about 10 μg/dose to about 100 μg/dose, or from about 100 μg/dose to about 1000 μg/dose.

A Type I or a Type III interferon receptor agonist can be administered daily, every other day, once a week, three times a week, every other week, three times per month, once monthly, substantially continuously or continuously.

In some embodiments, the Type I interferon receptor agonist is an IFN-α. Effective dosages of an IFN-α can range from about 1 μg to about 30 μg, from about 3 μg to about 27 μg, from about 1 MU to about 20 MU, from about 3 MU to about 10 MU, from about 90 μg to about 180 μg, or from about 18 μg to about 90 μg.

Effective dosages of Infergen® consensus IFN-α containing an amount of about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b can contain an amount of about 3 million Units (M) to about 10 MU, of drug per dose. Effective dosages of PEGASYS®PEGylated IPN-α2a can contain an amount of about 90 μg to 270 μg, or about 180 μg, of drug per dose. Effective dosages of PEG-INTRON®PEGylated IFN-α2b can contain an amount of about 0.5 μg to about 3.0 μg of drug per kg of body weight per dose. Effective dosages of PEGylated consensus interferon (PEG-CIFN) can contain an amount of about 18 μg to about 90 μg, or about 27 μg to about 60 μg, or about 45 μg, of CIFN amino acid weight per dose of PEG-CIFN. Effective dosages of monoPEG (30 kD, linear)-ylated CIFN can contain an amount of about 45 μg to about 270 μg, or about 60 μg to about 180 μg, or about 90 μg to about 120 μg, of drug per dose. In many embodiments, IFN-α or IFN-γ is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations. In some embodiments, the dosages are administered subcutaneously.

In carrying out the methods of combination therapy for smallpox virus infection in an individual as described above, a Type I or Type III interferon receptor agonist and a Type II interferon receptor agonist can be administered daily, twice daily, every other day, twice a week, three times a week, or substantially continuously or continuously. Effective dosages of a Type II interferon receptor agonist can range from about 1 μg to about 1000 μg. A Type II interferon receptor agonist can be administered daily, every other day, once a week, three times a week, every other week, three times per month, once monthly, substantially continuously or continuously.

In some embodiments, the Type II interferon receptor agonist is IFN-γ. Effective dosages of IFN-γ can range from about 25 μg/dose to about 300 μg/dose, from about 10 μg/dose to about 100 μg/dose, or from about 100 μg/dose to about 1000 μg/dose.

A Type I or a Type III interferon receptor agonist can be administered daily, every other day, once a week, three times a week, every other week, three times per month, once monthly, substantially continuously or continuously.

In some embodiments, the Type I interferon receptor agonist is an IFN-α. Effective dosages of an IFN-α can range from about 1 μg to about 200 μg, e.g., from about 1 μg to about 30 μg, from about 3 μg to about 27 μg, from about 1 MU to about 20 MU, from about 3 MU to about 10 MU, from about 90 μg to about 180 μg, or from about 18 μg to about 90 μg.

Effective dosages of Infergen® consensus IFN-α can contain an amount of about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b can contain an amount of about 3 million Units (MU) to about 10 MU of drug per dose. Effective dosages of PEGASYS®PEGylated IFN-α2a can contain an amount of about 90 μg to about 270 μg, or about 180 μg, of drug per dose. Effective dosages of PEG-INTRON®PEGylated IFN-α2b can contain an amount of about 0.5 μg to about 3.0 μg of drug per kg of body weight per dose. Effective dosages of PEGylated consensus interferon (PEG-CIFN) can contain an amount of about 18 μg to about 90 μg, or about 27 μg to about 60 μg, or about 45 μg, of CIFN amino acid weight per dose of PEG-CIFN.

Effective dosages of monoPEG (30 kD, linear)-ylated CIFN can contain an amount of about 45 μg to about 270 μg, or about 60 μg to about 180 μg, or about 90 μg to about 120 μg, of drug per dose. In many embodiments, IFN-α and/or IFN-γ is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

Dosage regimens can include tid, bid, qd, qod, biw, tiw, qw, qow, three times per month, or monthly administrations. In some embodiments, the invention provides any of the above-described methods in which the desired dosage of IFN-α is administered subcutaneously to the patient by bolus delivery qd, qod, tiw, biw, qw, qow, three times per month, or monthly, or is administered subcutaneously to the patient per day by substantially continuous or continuous delivery, for the desired treatment duration. In other embodiments, the invention provides any of the above-described methods in which the desired dosage of PEGylated IFN-α (PEG-IFN-α) is administered subcutaneously to the patient by bolus delivery qw, qow, three times per month, or monthly for the desired treatment duration.

In some embodiments, the invention provides methods using a synergistically effective amount of a Type I or Type III interferon receptor agonist and a Type II interferon receptor agonist in the treatment of smallpox virus infection in a patient. In some embodiments, the invention provides a method using a synergistically effective amount of an IFN-α and IFN-γ in the treatment of smallpox virus infection in a patient. In one embodiment, the invention provides a method using a synergistically effective amount of a consensus IFN-α and IF-γ in the treatment of smallpox virus infection in a patient.

In general, a synergistically effective amount of a consensus interferon (CIFN) and IFN-γ suitable for use in the methods of the invention is provided by a dosage ratio of 1 μg CIFN: 10 μg IFN-γ, where both CIFN and IFN-γ are unPEGylated and unglycosylated species.

In one embodiment, the invention provides a method using a synergistically effective amount of INFERGEN®consensus IFN-α and IFN-γ in the treatment of smallpox virus infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN®consensus IFN-α and IFN-γ in the treatment of smallpox virus infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN®consensus IFN-α and IFN-γ in the treatment of smallpox virus infection in a patient comprising administering to the patient a dosage of INFERGEN® containing an amount of about 1 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 50 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN®consensus IFN-α and IFN-γ in the treatment of smallpox virus infection in a patient comprising administering to the patient a dosage of INFERGEN® containing an amount of about 9 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 90 μg to about 100 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of INFERGEN® consensus IFN-α and IFN-γ in the treatment of smallpox virus infection in a patient comprising administering to the patient a dosage of INFERGEN® containing an amount of about 30 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 200 μg to about 300 μg of drug per dose of IFN-γ, subcutaneously qd, qod, tiw, biw, qw, qow, three times per month, once monthly, or per day substantially continuously or continuously, for the desired treatment duration.

In another embodiment, the invention provides a method using a synergistically effective amount of PEGylated consensus IFN-α and IFN-γ in the treatment of smallpox virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a total weekly dosage of IFN-γ containing an amount of about 30 μg to about 1,000 μg of drug per week in divided doses administered subcutaneously qd, qod, tiw, or biw, or substantially continuously or continuously, for the desired treatment duration.

In another embodiment, the invention provides a method using a synergistically effective amount of PEGylated consensus IFN-α and IFN-γ in the treatment of smallpox virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a total weekly dosage of IFN-γ containing an amount of about 100 μg to about 300 μg of drug per week in divided doses administered subcutaneously qd, qod, tiw, biw, or substantially continuously or continuously, for the desired treatment duration.

In general, a synergistically effective amount of IFN-α2a or 2b or 2c and IFN-γ suitable for use in the methods of the invention is provided by a dosage ratio of 1 million Units (MU) IFN-α2a or 2b or 2c: 30 μg IFN-γ, where both IFN-α2a or 2b or 2c and IFN-γ are unPEGylated and unglycosylated species.

In another embodiment, the invention provides a method using a synergistically effective amount of IFN-α2a or 2b or 2c and IFN-γ in the treatment of smallpox virus infection in a patient comprising administering to the patient a dosage of IFN-α2a containing an amount of about 1 MU to about 20 MU of drug per dose of IFN-α2a, 2b or 2c subcutaneously qd, qod, tiw, biw, or per day substantially continuously or continuously, in combination with a dosage of IFN-γ containing an amount of about 30 μg to about 600 μg of drug per dose of IFN-γ, subcutaneously qd, qod, tiw, biw, or per day substantially continuously or continuously, for the desired treatment duration.

In another embodiment, the invention provides a method using a synergistically effective amount of IFN-α2a or 2b or 2c and IFN-γ in the treatment of smallpox virus infection in a patient comprising administering to the patient a dosage of IFN-α2a containing an amount of about 3 MU of drug per dose of IFN-α2a, 2b or 2c subcutaneously qd, qod, tiw, biw, or per day substantially continuously or continuously, in combination with a dosage of IFN-γ containing an amount of about 100 μg of drug per dose of IFN-γ, subcutaneously qd, qod, tiw, biw, or per day substantially continuously or continuously, for the desired treatment duration.

In another embodiment, the invention provides a method using a synergistically effective amount of IFN-α2a or 2b or 2c and IFN-γ in the treatment of smallpox virus infection in a patient comprising administering to the patient a dosage of IFN-α2a containing an amount of about 10 MU of drug per dose of IFN-α2a, 2b or 2c subcutaneously qd, qod, tiw, biw, or per day substantially continuously or continuously, in combination with a dosage of IFN-γ containing an amount of about 300 μg of drug per dose of IFN-γ, subcutaneously qd, qod, tiw, biw, or per day substantially continuously or continuously, for the desired treatment duration.

In another embodiment, the invention provides a method using a synergistically effective amount of PEGASYS®PEGylated IFN-α2a and IFN-γ in the treatment of smallpox virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a total weekly dosage of IFN-γ containing an amount of about 30 μg to about 1,000 μg, of drug per week administered in divided doses subcutaneously qd, qod, tiw, or biw, or administered substantially continuously or continuously, for the desired treatment duration.

In another embodiment, the invention provides a method using a synergistically effective amount of PEGASYS®PEGylated IFN-α2a and IFN-γ in the treatment of smallpox virus infection in a patient comprising administering to the patient a dosage of PEGASYS® containing an amount of about 180 μg of drug per dose of PEGASYS®, subcutaneously qw, qow, three times per month, or monthly, in combination with a total weekly dosage of IFN-γ containing an amount of about 100 μg to about 300 μg, of drug per week administered in divided doses subcutaneously qd, qod, tiw, or biw, or administered substantially continuously or continuously, for the desired treatment duration.

In another embodiment, the invention provides a method using a synergistically effective amount of PEG-INTRON®PEGylated IFN-α2b and IFN-γ in the treatment of smallpox virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a total weekly dosage of IFN-γ containing an amount of about 30 μg to about 1,000 μg of drug per week administered in divided doses subcutaneously qd, qod, tiw, or biw, or administered substantially continuously or continuously, for the desired treatment duration.

In another embodiment, the invention provides a method using a synergistically effective amount of PEG-INTRON®PEGylated IFN-α2b and IFN-γ in the treatment of smallpox virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a total weekly dosage of IFN-γ containing an amount of about 100 μg to about 300 μg of drug per week administered in divided doses subcutaneously qd, qod, tiw, or biw, or administered substantially continuously or continuously, for the desired treatment duration.

The invention also provides methods for the treatment of smallpox virus infection in which therapy with an additional antiviral agent is added to any of the Type I or Type III interferon receptor agonist and a Type II interferon receptor agonist monotherapies or combination therapies described above.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a poxvirus infection, the method comprising administering to an individual an effective amount of IFN-α.

2. The method of claim 1, wherein the IFN-α is administered concurrently with a vaccinia virus vaccine.

3. The method of claim 1, wherein the individual has been vaccinated with vaccinia virus.

4. The method of claim 3, wherein the IFN-α is administered from 1 to 30 days after vaccination.

5. The method of claim 1, wherein IFN-α is administered from 1 day to 1 year before administration of a vaccinia virus vaccine.

6. The method of claim 1, further comprising administering a vaccinia virus vaccine.

7. The method of claim 1, wherein the individual has been exposed to smallpox virus, and the IFN-α is administered within 24 hours of exposure to smallpox virus.

8. The method of claim 1, wherein the individual has been exposed to smallpox virus, and the IFN-α is administered within 48 hours of exposure to smallpox virus.

9. The method of claim 1, wherein the individual has been exposed to smallpox virus, and the IFN-α is administered 72 hours to 35 days after exposure to smallpox virus.

10. A method of treating a poxvirus infection, the method comprising administering to an individual an effective amount of IFN-γ.

11. The method of claim 10, wherein the IFN-γ is administered concurrently with a vaccinia virus vaccine.

12. The method of claim 10, wherein the individual has been vaccinated with vaccinia virus.

13. The method of claim 12, wherein the IFN-γ is administered from 1 to 30 days after vaccination.

14. The method of claim 10, wherein IFN-γ is administered from 1 day to 1 year before administration of a vaccinia virus vaccine.

15. The method of claim 10, further comprising administering a vaccinia virus vaccine.

16. The method of claim 10, wherein the individual has been exposed to smallpox virus, and the IFN-γ is administered within 24 hours of exposure to smallpox virus.

17. The method of claim 10, wherein the individual has been exposed to smallpox virus, and the IFN-γ is administered within 48 hours of exposure to smallpox virus.

18. The method of claim 10, wherein the individual has been exposed to smallpox virus, and the IFN-γ is administered 72 hours to 35 days after exposure to smallpox virus.

19. A method of treating a poxvirus infection, the method comprising administering to an individual effective amounts of IFN-γ and IFN-α.

20. The method of claim 19, wherein the IFN-γ and IFN-α are administered concurrently with a vaccinia virus vaccine.

21. The method of claim 19, wherein the individual has been vaccinated with vaccinia virus.

22. The method of claim 21, wherein the IFN-γ and IFN-α are administered from 1 to 30 days after vaccination.

23. The method of claim 19, wherein the IFN-γ and IFN-α are administered from 1 day to 1 year before administration of a vaccinia virus vaccine.

24. The method of claim 19, further comprising administering a vaccinia virus vaccine.

25. The method of claim 19, wherein the individual has been exposed to smallpox virus, and the IFN-γ and IFN-α are administered within 24 hours of exposure to smallpox virus.

26. The method of claim 19, wherein the individual has been exposed to smallpox virus, and the IFN-γ and IFN-α are administered within 48 hours of exposure to smallpox virus.

27. The method of claim 19, wherein the individual has been exposed to smallpox virus, and the IFN-γ and IFN-α are administered 72 hours to 35 days after exposure to smallpox virus.

28. The method of claim 1, further comprising administering an effective amount of a nucleotide analog or a nucleoside analog.

29. The method of claim 1, wherein the IFN-α is a consensus interferon.

30. The method of claim 19, wherein the IFN-α is a consensus interferon.

31. The method of claim 10, further comprising administering an effective amount of a nucleotide analog or a nucleoside analog.

32. The method of claim 19, further comprising administering an effective amount of a nucleotide analog or a nucleoside analog.
